(19) United States
(12) Patent Application Publication
(10) Pub. No.: US 2014/0288421 A1
(43) Pub. Date: Sep. 25, 2014

(54) GAS VESICLE ULTRASOUND CONTRAST AGENTS AND METHODS OF USING THE SAME

(71) Applicant: The Regents of the University of California, Oakland, CA (US)

(72) Inventors: Mikhail Georgievich Shapiro, San Francisco, CA (US); Patrick William Goodwill, Mountain View, CA (US); David Vernon Schaffer, Danville, CA (US); Steven Michael Conolly, Palo Alto, CA (US)

(21) Appl. No.: 14/206,698

(22) Filed: Mar. 12, 2014

Related U.S. Application Data
(60) Provisional application No. 61/778,095, filed on Mar. 12, 2013.

(51) Int. Cl. A61B 8/08; A61K 49/22

(52) U.S. Cl. CPC A61B 8/481; A61B 8/5238; A61B 8/5207; A61K 49/223
USPC 600/431; 424/9.52

(57) ABSTRACT

Provided are ultrasound imaging methods that include administering to a subject a contrast agent that includes a plurality of collapsible gas vesicles, obtaining ultrasound data of a target site of interest, and analyzing the ultrasound data to produce an ultrasound image of the target site. Ultrasound contrast agents are also provided. The subject methods and contrast agents find use in ultrasound imaging applications.

### CROSS-REFERENCE TO RELATED APPLICATIONS

Pursuant to 35 U.S.C. §119 (e), this application claims priority to the filing date of the U.S. Provisional Patent Application Ser. No. 61/778,095 filed Mar. 12, 2013; the disclosure of which application is herein incorporated by reference.

### REFERENCE TO GOVERNMENT SUPPORT

This invention was made with government support under grant number R01 ES020903 awarded by the National Institutes of Health. The Government has certain rights in this invention.

### INTRODUCTION

Ultrasound is among the most widely used imaging modalities in biomedicine due to its superior spatial and temporal resolution and lower cost compared to other non-invasive imaging techniques. However, ultrasound plays a minor role in molecular imaging due to a lack of suitable molecular reporters. Contrast-enhanced ultrasound (CEUS) is the application of an ultrasound contrast agent to traditional medical sonography. Ultrasound contrast agents interact with sound waves so as to produce acoustic signals, such as scattering and attenuation, that can be detected by imaging equipment. Ultrasound contrast agents may have a high degree of echogenicity, which is the ability of an object to reflect the ultrasound waves. The echogenicity difference between the ultrasound contrast agent and the soft tissue surroundings of the body enhances the ultrasound backscatter, or reflection of the ultrasound waves, to produce a sonogram with increased contrast due to the high echogenicity difference. Contrast-enhanced ultrasound can be used to image blood perfusion in organs, measure blood flow rate in the heart and other organs, and has other applications as well.

### SUMMARY

Provided are ultrasound imaging methods that include administering to a subject a contrast agent that includes a plurality of collapsible gas vesicles, obtaining ultrasound data of a target site of interest, and analyzing the ultrasound data to produce an ultrasound image of the target site of the subject. Ultrasound contrast agents are also provided, which include a plurality of collapsible gas vesicles. The subject methods and contrast agents find use in ultrasound imaging applications.

### BRIEF DESCRIPTION OF THE FIGURES

FIG. 1a shows a diagram of a gas vesicle (GV) that includes a hollow gas nanocompartment surrounded by a gas-permable protein shell, according to embodiments of the present disclosure. FIG. 1b shows transmission electron microscopy (TEM) images of intact (left) and collapsed (right) Anabaena flos-aquae (Ana) GVs. FIG. 1c shows TEM images of intact (left) and collapsed (right) Halobacterium NRC-1 (Halo) GVs. Scale bars are 200 nm. FIG. 1d shows ultrasound images of an ultrasound phantom containing Ana GVs at different optical densities, collapsed Ana GVs (“Col.”, OD 2.0) or buffer (phosphate buffered saline, PBS). Images were acquired at multiple frequencies, as indicated. FIG. 1e shows ultrasound images of Halo GVs arranged as described for FIG. 1d. FIG. 1f shows a graph of total backscattered signal relative to PBS at each frequency and Ana GV concentration (N=4 per sample). FIG. 1g shows a graph of total backscattered signal relative to PBS at each frequency and Halo GV concentration (N=4 per sample). Detailed image acquisition and analysis parameters are provided in Table 1; color bars in FIG. 8.

FIG. 2a shows a graph of the power spectrum of signal backscattered from Halo GVs (black) and 4.78 μm polystyrene (PS) microspheres (gray) in response to 6 MHz pulses, according to embodiments of the present disclosure. The shaded highlights in FIG. 2a correspond to frequency bands used to generate the images in FIG. 2b. FIG. 2b shows images of Halo GVs and PS microspheres acquired with 6 MHz transmission and band-pass filtered around 6, 12 and 18 MHz. FIG. 2c shows a graph of the ratio of total backscattered signal from Halo GVs and PS microspheres after filtering at the indicated frequencies (N=4). FIG. 2d shows images of Halo GVs, Ana GVs and PS micro-spheres at 8.6 MHz acquired before (Pre) and after (Post) destructive collapse with 650 kPa insonation, and the difference (Diff.) between these images. FIG. 2e shows a graph of the ratio of total backscattered signal from GVs and PS microspheres in pre-collapse and difference images (N=4). The concentrations used in FIGS. 2a-e were OD 0.5 Halo GVs, OD 2.0 Ana GVs and 0.83% w/v polystyrene. FIG. 2f shows images of an ultrasound phantom containing Ana GVs, Halo GVs, a mixture of both (all GVs at OD 1.0) and PBS, acquired at 8.6 MHz. Top: before collapse. Second from top: pre-collapse image subtracted by an image acquired after 300 kPa collapse. Third from top: 300 kPa-collapsed image subtracted by an image acquired after 650 kPA collapse. Bottom: overlay of the two middle images. Detailed image acquisition and analysis parameters are provided in Table 1; color bars in FIG. 8.

FIG. 3a shows a diagram of predicted aggregation interactions between surface-biotinylated GVs (hexagons with gray arrows) and streptavidin (SA) at different SA:GV ratios, according to embodiments of the present disclosure. FIG. 3b shows a 17 MHz image of OD 1.0 biotinylated GVs mixed with the indicated ratio of SA. FIG. 3c shows a graph of integrated signal intensity relative to phantom background corresponding to the SA:GV conditions in FIG. 3b (N=4 per condition). FIG. 3d shows TEM images of GVs incubated with SA at the indicated molar ratios. At the higher magnification (right), arrows indicate apparent SA molecules on the GV surface. Scale bars are 2 μm and 40 nm, respectively. FIG. 3e shows a diagram of GVs (black hexagons) confined inside intact cells (circles) or released following lysis. FIG. 3f shows an image of Ana cells treated with water (intact) or with 50% sucrose (lysed) at 17 MHz. FIG. 3g shows a graph of integrated signal intensity relative to phantom background for intact and lysed cells (N=4 per condition). Detailed image acquisition and analysis parameters are provided in Table 1; color bars in FIG. 8.

FIG. 4a shows an image overlay of second harmonic image (6 MHz pulses) in a grayscale broadband anatomical image of mouse lower abdomen injected subcutaneously with 150 μL OD 6.0 Halo GVs on the right side and 150 μL PBS on the left side, according to embodiments of the present disclosure. FIGS. 4b-c shows images of a second harmonic image before (FIG. 4b) and after (FIG. 4c) GV collapse with destructive insonation (650 kPa). Dashed ovals indicate approximate location of each injection. Detailed image acquisition and analysis parameters are provided in Table 1; color bars in FIG. 8.

FIG. 5a shows an ultrasound image (17 MHz) of a phantom containing OD 2.0 Ana GVs and Halo GVs or PBS, acquired repeatedly over several days, according to embodiments of the present disclosure. The intensity profile of Ana GVs shows minimal change. Halo GV signal appears to show less of a shadowing effect, which may be due to a decrease in Halo GV concentration. FIG. 5b shows a graph of total backscattered signal from Halo and Ana GVs on each day of sampling, relative to PBS control (N=4/sample). Detailed image acquisition and analysis parameters are provided in Table 1; color bars in FIG. 8.

FIG. 6 shows an ultrasound image (17 MHz) of phantom containing Halo GVs at indicated optical densities, collapsed OD 2.0 Halo GVs (Col.) or buffer (PBS), according to embodiments of the present disclosure. The intense signal at the bottom of the image corresponds to the solid bottom of the imaging phantom. The arrow indicates a location underneath Halo GVs where the signal from the solid bottom was attenuated. Smaller attenuation can be observed at lower concentrations of GVs. Detailed image acquisition and analysis parameters are provided in Table 1; color bars in FIG. 8.

FIG. 7 shows ultrasound images of three mice that were depilated and injected subcutaneously with 150 μL OD 6.0 Halo GVs or 150 μL PBS on opposite sides of the abdomen, according to embodiments of the present disclosure. FIGS. 7a, d and g show anatomical images acquired with 10 MHz transducer. FIGS. 7b, e and h show 12 MHz harmonic images (6 MHz pulsing). FIGS. 7c, f and i show 12 MHz harmonic images after GV collapse with destructive insonation. Dashed ovals indicate approximate location of injected material. Subject 1 is also shown in FIG. 4. Detailed image acquisition and analysis parameters are provided in Table 1; color bars in FIG. 8.

FIG. 8 shows color maps used in the images; Gray (left), Hot (second from left), Green (third from left) and Magenta (right) color maps, according to embodiments of the present disclosure. The scales are linear between Min and Max. The color map used and the values of Min and Max are defined for each image in Table 1.

### DETAILED DESCRIPTION

Provided are ultrasound imaging methods that include administering to a subject a contrast agent that includes a plurality of collapsible gas vesicles, obtaining ultrasound data of a target site of interest, and analyzing the first ultrasound data to produce an ultrasound image of the target site. Ultrasound contrast agents are also provided, which include a plurality of collapsible gas vesicles. The subject methods and contrast agents find use in ultrasound imaging applications.

Below, the subject ultrasound contrast agents are described first in greater detail. Ultrasound imaging methods are also disclosed in which the subject ultrasound contrast agents find use. In addition, multiplex ultrasound imaging methods and kits that include the subject ultrasound contrast agents are also described.

### Ultrasound Contrast Agents

Embodiments of the present disclosure include an ultrasound contrast agent. The ultrasound contrast agent may be configured to increase contrast in ultrasound images of a subject. By an increase in contrast is meant that differences in image intensity between adjacent tissues visualized by ultrasound are enhanced. For instance, differences in image intensity may be enhanced with the use one or more sets of imaging parameters. In certain embodiments, the ultrasound contrast agent includes gas vesicles (GVs), such as a plurality of gas vesicles. In certain embodiments, the gas vesicles are genetically encoded gas vesicles. For example, the gas vesicles may be microorganism derived, such as bacterially-derived, gas vesicles formed by bacteria, such as photosynthetic bacteria (e.g., cyanobacteria), or the gas vesicles may be archaea-derived gas vesicles formed by archaea (e.g., halobacteria).

Gas vesicles may be derived from any number of species of microorganism, e.g., bacteria or archaea. For example, gas vesicles may be derived from various prokaryotes, including cyanobacteria such as Microcystis aeruginosa, Aphanizomenon flos aquae and Oscillatoria agardhii; phototropic bacteria such as Amoebobacter, Thiodictyon, Pelodictyon, and Ancalochloris; nonphototropic bacteria such as Microcyclus aquaticus; Gram-positive bacteria such as Bacillus megaterium; Gram-negative bacteria such as Serratia; and archaea such as Haloferax mediterranei, Methanosarcina barkeri, Halobacteria salinarium.

In certain embodiments, the gas vesicles are isolated from bacteria or archaea using any convenient method known in the art (See, e.g., Sremac et al., 2008. BMC Biotech 8:9; U.S. Pat. No. 7,022,509; the disclosures of each of which are incorporated herein by reference). In certain instances, gas vesicles are isolated by centrifugally-assisted flotation following cell lysis. In certain instances, aggregation of gas vesicles using flocculating agents (such as polyethylenimines, polyacrylamides, polyamine derivatives, ferric chloride, and alum) enhance the buoyancy of gas vesicles and facilitate isolation of gas vesicles.

In certain embodiments, the gas vesicles are substantially spherical in shape. In some instances, the gas vesicles are ellipsoid in shape. Other shapes are also possible depending on the type of bacteria the gas vesicles are derived from. For instance, the gas vesicles may be cylindrical in shape, or may have a center portion that is cylindrical with end portions that are cone shaped, or may be football shaped, and the like.

In certain embodiments, the gas vesicles have dimensions that are nanoscale, with exact sizes and shapes varying between genetic hosts. By nanoscale is meant that the average dimensions of the gas vesicles are 1000 nm or less, such as 900 nm or less, including 800 nm or less, or 700 nm or less, or 600 nm or less, or 500 nm or less, or 400 nm or less, or 300 nm or less, or 250 nm or less, or 200 nm or less, or 150 nm or less, or 100 nm or less, or 75 nm or less, or 50 nm or less, or 25 nm or less, or 10 nm or less. For example, the average diameter of the gas vesicles may range from 10 nm to 1000 nm, such as 25 nm to 500 nm, including 50 nm to 250 nm, or 100 nm to 250 nm. By “average” is meant the arithmetic mean.

In certain embodiments, the gas vesicle has a vesicle wall. The vesicle wall may be produced by the bacteria the gas vesicles are derived from. For instance, the gas vesicles may have a vesicle wall that is composed of a protein or peptide. In certain cases, the vesicle wall is a gas-permeable vesicle wall. In these instances, the vesicle wall may be permeable to a gas (e.g., air, oxygen, nitrogen, noble gases, such as helium, neon, argon, krypton, xenon, dodecafluoropentane), but is substantially impermeable to aqueous media (e.g., water, saline, buffer, the surrounding liquid media the contrast agent is in during use, etc.). As such, a gas (e.g., a gas from the surrounding media) may substantially freely diffuse in and out of the gas vesicles, whereas liquids are substantially excluded from the interior of the gas vesicles. In these embodiments, substantially no pressure gradient exists between the inside and outside of gas vesicles, which in some cases may facilitate the stability of the structure of the gas vesicles.

In certain embodiments, the gas vesicles have an interior volume of 5 picoliters (pL) or less, such as 1 pL or less, including 750 femtoliters (fL) or less, or 500 fL or less, or 250 fL or less, or 100 fL or less, or 75 fL or less, or 50 fL or less, or 25 fL or less, or fL or less, or 1 fL or less, or 750 attoliters (aL) or less, or 500 aL or less, or 250 aL or less, or 100 aL or less, or 75 aL or less, or 50 aL or less, or 25 aL or less, or 10 aL or less, or 5 aL or less, or 1 aL or less.

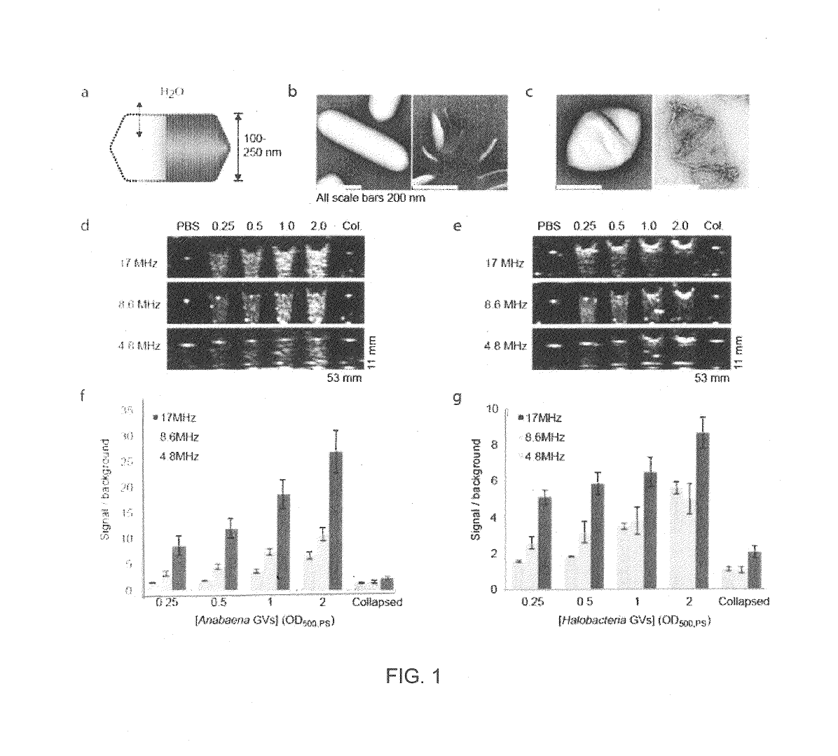

In certain embodiments, the vesicle wall is configured to maintain the shape and size of the gas vesicles under normal usage conditions (e.g., production, isolation, storage, administration to a subject, imaging). In certain instances, the vesicle wall has a thickness of 10 nm or less, such as 9 nm or less, including 8 nm or less, or 7 nm or less, or 6 nm or less, or 5 nm or less, or 4 nm or less, or 3 nm or less, or 2 nm or less, or 1 nm or less. For example, FIG. 1a shows a diagram of a gas vesicle (GV) that includes a hollow gas nanocompartment surrounded by a semipermeable protein shell.

In certain embodiments, the gas vesicles include a specific binding moiety attached to a surface of the gas vesicles. The specific binding moiety may be configured to specifically bind to a target site in a subject. For example, the specific binding moiety may include a binding member stably associated with a surface of the gas vesicle. By “stably associated” is meant that a moiety is bound to or otherwise associated with another moiety or structure under standard conditions. In certain instances, the specific binding moiety is stably associated with (e.g., bound to) a surface of the gas vesicle, as described above. Bonds may include covalent bonds and non-covalent interactions, such as, but not limited to, ionic bonds, hydrophobic interactions, hydrogen bonds, van der Waals forces (e.g., London dispersion forces), dipole-dipole interactions, and the like. In certain embodiments, the specific binding moiety may be covalently bound to the gas vesicle. Covalent bonds between the specific binding moiety and the gas vesicle include covalent bonds that involve reactive groups, such as, but not limited to, N-hydroxysuccinimide (NHS) esters (such as sulfo-NHS esters), imidoesters, aryl azides, diazirines, carbodiimides, maleimides, cyanates, iodoacetamides, and the like.

A binding moiety can be any molecule that specifically binds to a target site of interest, e.g., protein, peptide, biomacromolecule, cell, tissue, etc. that is being targeted. In some embodiments, the affinity between a binding moiety and its target site to which it specifically binds when they are specifically bound to each other in a binding complex is characterized by a K_{D} (dissociation constant) of 10⁻⁵ M or less, 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less. “Affinity” refers to the strength of binding, increased binding affinity being correlated with a lower K_{D}.

The specific binding moiety can be any molecule that specifically binds to a protein, peptide, biomacromolecule, cell, tissue, etc. that is being targeted (e.g., a protein peptide, biomacromolecule, cell, tissue, etc. at a target site of interest in a subject). Depending on the nature of the target site, the specific binding moiety can be, but is not limited to, an antibody against an epitope of a peptidic analyte, or any recognition molecule, such as a member of a specific binding pair. For example, suitable specific binding pairs include, but are not limited to: a member of a receptor/ligand pair; a ligand-binding portion of a receptor; a member of an antibody/antigen pair; an antigen-binding fragment of an antibody; a hapten; a member of a lectin/carbohydrate pair; a member of an enzyme/substrate pair; biotin/avidin; biotin/streptavidin; digoxin/antidigoxin; a member of a peptide aptamer binding pair; and the like.

In certain embodiments, the specific binding moiety includes an antibody. An antibody as defined here may include fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may also include Fab′, Fv, F(ab′)₂, and or other antibody fragments that retain specific binding to antigen.

In certain embodiments, the antibody may specifically bind to an analyte at the target site of interest. In some cases, the specific binding moiety is stably associated with a gas vesicle, as described above. The gas vesicle-bound specific binding moiety may be configured to specifically bind to an analyte at a target site of interest in a subject. As such, specific binding of the gas vesicle-bound specific binding moiety to the analyte at the target site of interest may indirectly bind the gas vesicle to the target site of interest in the subject. Binding of the gas vesicle to the target site may stably associate the gas vesicle with the target site and thus facilitate detection of the ultrasound contrast agent containing the gas vesicles and thus facilitate the production of an ultrasound image of the target site of interest in the subject.

In certain embodiments, the binding moiety is a bifunctional binding moiety, such as a bifunctional crosslinking agent. Bifunctional crosslinking agents may include, but not limited to, bisdiazobenzidine, N,N′-ethylene bismaleimaide, hexamethylene diisocyanate, toluene diisocyanate, hexamethryline diisothiocyanate, N,N′-polymethylene bisiodoacetamide, and glutaraldehyde.

In certain instances, the gas vesicle-bound specific binding moiety is configured to bind other gas vesicles. In these instances, gas vesicles bind to each other via the specific binding moieties to form aggregates of gas vesicles. For example, the specific binding moiety is attached to a surface of the gas vesicles and configured to form aggregates of the gas vesicles. In certain embodiments, the specific binding moiety is sulfo-NHS-LC-Biotin that is attached to a surface of the gas vesicles and configured to bind to other gas vesicles attached to sulfo-NHS-LC-Biotin in the presence of streptavidin. For example, addition of an appropriate amount of streptavidin to a solution containing gas vesicles functionalized with sulfo-NHS-LC-Biotin induces the formation of gas vesicle aggregates. In certain embodiments, the specific binding moiety is a bifunctional crosslinking agent configured to form aggregates of gas vesicles.

In certain embodiments, the gas vesicles form aggregates with an average diameter of 80 nm or greater, or 120 nm or greater, or 160 nm or greater, or 200 nm or greater, or 250 nm or greater, or 300 nm or greater, or 400 nm or greater, or 500 nm or greater, or 1000 nm or greater. For example, the average diameter of the gas vesicle aggregate is about 1000 nm.

In certain embodiments, the gas vesicle is a collapsible gas vesicle. By collapsible gas vesicle is meant a gas vesicle configured such that the vesicle wall of the gas vesicle may be disrupted by the application of an external force, such as an externally applied pressure. For example, under normal use conditions (e.g., production, isolation, storage, administration to a subject, imaging), the gas vesicle may be configured to be substantially stable, such that the physical structure of the gas vesicle is not substantially disrupted. Stated another way, the physical structure of the gas vesicle maintains substantially the same integrity and water exclusion under normal use conditions. In some instances, the vesicle wall of the gas vesicle may be disrupted by application of an external force, such as an external pressure. For example, application of an externally applied pressure may disrupt the semipermeable integrity of the vesicle wall such that the vesicle wall is substantially permeable to the surrounding media (e.g., fluids, such as water, saline, buffer, the surrounding fluid media when the contrast agent is in during use, etc.). As such, a collapsed gas vesicle may provide a contrast in an ultrasound image that is substantially less than the contrast provided by an intact gas vesicle. For instances, a collapsed gas vesicle may provide substantially no contrast enhancement as compared to an intact gas vesicle. In some cases, the externally applied pressure may be generated by applying ultrasound waves (e.g., an ultrasonic pulse) to the gas vesicles sufficient to produce an increase in pressure at the target site of interest where the gas vesicles are located.

In certain embodiments, the contrast agent includes one or more cells containing intracellular gas vesicles. These gas vesicles may be loaded into cells as part of contrast agent preparation, or may be expressed by cells based on gas vesicle encoding genes contained within the cells. Methods of loading gas vesicles into cells may include any convenient method known in the art, including, but not limited to, chemical transfection (calcium phosphate transfection, lipofection, cationic polymer transfection) electroporation, particle bombardment, cell-penetrating peptide-mediated transport, and the like. For example, cell-penetrating peptides (such as TAT, RGD, and Rabies virus-derived peptide) are used to deliver nanoparticles greater than 300 nm into cells (See., e.g., Delehanty et al., 2010. Ther Deliv 1:411; the disclosure of which is incorporated herein by reference).

Genes required for gas vesicle formation may be found in any number of species of naturally occurring bacteria or archaea that produce gas vesicles, as described above. In certain instances, gas vesicle-forming genes may be obtained from these bacteria or archaea. In certain instances, the gas vesicle-forming genes comprise genes that encode proteins that form part of the gas vesicles and/or genes that regulate the expression of genes that encode the gas vesicle proteins. In some instances, the genes required for gas vesicle formation are found in a cluster in the genome of the bacteria or archaea species. The cluster of genes may include a gene encoding for a single highly conserved protein, GvpA, or a closely related homolog thereof, such as GvpB found in Bacillus megaterium. In certain instances, GvpA is the primary protein component of gas vesicles and when assembled forms a hydrophobic inner surface of the gas vesicle and a hydrophilic exterior surface. An exemplary amino acid sequence of GvpA from Anabaena flos-aquae (GID 3683431) is shown below.

(SEQ ID NO: 1)
MAVEKTNSSSSLAEVIDRILDKGIVVDAWVRVSLVGIELLAIEARIVIAS
VETYLKYAEAVGLTQSAAMPA

In certain instances, a 6 kilobase (kb) cluster encoding 11 gas vesicle genes from Bacillus megaterium may be sufficient to confer formation of gas vesicles when heterologously expressed in E. coli. In yet another instance, a 16.6 kb cluster encoding GV genes from Serratia sp. may be sufficient to confer formation of gas vesicles when heterologously expressed in E. coli. The genes contained in these gas vesicle gene clusters are listed in the table below.

Genes in Gas Vesicle Gene Clusters that are Sufficient to Induce Gas Vesicle Formation when Expressed in E. coli

| Species/Strain | Gene name | GID |
|---|---|---|
| Bacillus megaterium | gvpB | 8987738 |
| | gvpR | 8987737 |
| | gvpN | 8987736 |
| | gvpF | 8987735 |
| | gvpG | 8987734 |
| | gvpL | 8987733 |
| | gvpS | 8987732 |
| | gvpK | 8987731 |
| | gvpJ | 8987730 |
| | gvpT | 8987729 |
| | gvpU | 8987728 |
| | araC | 8987727 |
| Serratia sp. ATCC strain | gvpA1 | 16810365 |
| 39006 | gvpC | 16810366 |
| | gvpN | 16810367 |
| | gvpV | 16810368 |
| | gvpF1 | 16810370 |
| | gvpG | 16810371 |
| | gvpW | 16810372 |
| | gvpA2 | 16810373 |
| | gvpK | 16810374 |
| | gvpX | 16810375 |
| | gvpA3 | 16810376 |
| | gvpY | 16810377 |
| | gvrA | 16810378 |
| | gvpH | 16810379 |
| | gvpZ | 16810380 |
| | gvpF2 | 16810381 |
| | gvpF3 | 16810382 |
| | gvrB | 16810383 |
| | gvrC | 16810384 |

In some embodiments, the cells are of a type that naturally produce gas vesicles. In some embodiments, the cells are bacterial cells heterologously expressing gas vesicles from a plasmid or genome-integrated DNA. “Heterologous,” in the context of two things that are heterologous to one another, refers to two things that do not exist in the same arrangement in nature. In the context of heterologous expression of genes or proteins, genes or proteins are heterologously expressed in a bacterial cell if the genes or proteins are not expressed in a naturally occurring bacterial cell. In some embodiments, the cells are eukaryotic cells, such as mammalian cells, heterologously expressing gas vesicles from a plasmid, viral vector or genome-integrated DNA. In these instances, genes or proteins are heterologously expressed in eukaryotic cells if the genes or proteins are not expressed in naturally occurring eukaryotic cells, such as mammalian cells. Methods for facilitating heterologous expression of prokaryotic genes in mammalian cells are known, including, but not limited to, codon optimization and polycistronic expression (See, e.g., Jinek et al., 2013. Elife 2:e00471; Close et al., 2010. PLoS One 5:e12441; Grohmann et al., 2009. BMC Cancer 9:301; the disclosures of each of which are incorporated herein by reference). In certain instances, mammalian cells heterologously expressing gas vesicles may be autologous or heterologous to the target individual. Such mammalian cells may be, for example, tumor cells, immune cells, stem cells or other cell types.

In certain embodiments, the gas vesicles are encoded genetically in one or more gene vectors, such as a non-viral gene delivery vector, a DNA virus or a RNA virus, and the gene vector or vectors are administered to the subject. Viral gene delivery vectors include, but are not limited to, adenoviruses, adeno-associated viruses, retroviruses and lentiviruses, as well as engineered combinations of natural viral variants, such as pseudotyped, mosaic or chimeric viral vectors. The gene vector may transfect all cells in the area of administration, or may target specific cells based on the characteristics of the vectors. In some embodiments, the vector is designed with promoters such that only a subset of transfected cells, or only under certain intracellular conditions, the gas vesicles are expressed in the target cells.

In certain embodiments, the heterologous expression of gas vesicles from a plasmid, viral vector or genome-integrated DNA is constant, or expression is only under certain times or under certain environmental conditions. In certain embodiments, expression is induced by a specific cue administered to the subject. For example, the specific cue may be a chemical inducer, temperature change, electromagnetic radiation, and the like. For example, expression of gas vesicles may be induced by IPTG, tetracycline, natural and synthetic steroid hormones, and the like. In certain embodiments, gas vesicle genes are integrated into the genome of a model organism, such that they are expressed in all or a subset of cells in that organism, constantly or at certain times or under certain conditions. In some embodiments, such an organism may me a transgenic mouse, zebrafish, or other species. Methods of integrating genes into the genome of a model organism may include any convenient method of gene targeting known in the art, including, but not limited to, viral integration, gamma-ray irradiation, Zinc-finger nuclease-mediated recombination, TALEN-mediated recombination, CRISPR/Cas-mediated recombination, Cre-Lox recombination, FLP-FRT recombination, PhiC31 integrase-mediated recombination, YR-mediated recombination, SR-mediated recombination, and the like.

### Ultrasound Imaging Methods

Embodiments of the methods are directed to ultrasound imaging methods. In certain instances, the method includes imaging a target site using an ultrasound protococol and a contrast agent such as described above, e.g., a contrast agent that includes a plurality of collapsible gas vesicles. A target site imaged by methods of invention may vary widely, and may be an in vitro or in vivo target site. As such, a target site may include, for example, any molecule, cell, tissue, body part, body cavity, organ system, whole organisms, collection of any number of organisms, etc., that are of interest. For example, target sites may include a solution comprising a collection of organisms, including, bacteria or archaea. In certain instances, target sites may include a solution comprising cells grown in culture, including, primary mammalian cells, immortalized cell lines, tumor cells, stem cells, and the like. In certain embodiments, target sites of interest include tissue and organs in culture. In certain embodiments, target sites of interest include tissue, organs, or organs systems in a subject, for example, lungs, brain, kidney, liver, heart, the central nervous system, the peripheral nervous system, the gastrointestinal system, the circulatory system, the immune system, the skeletal system, the sensory system, and the like.

In certain embodiments, administering the contrast agent includes administering the contrast agent produced and prepared outside the subject. In certain embodiments, administering the contrast agent includes administering to the subject one or more gene vectors that contain genes that encode the contrast agent, as described above.

In certain embodiments, the contrast agent is administered to a subject in any pharmaceutically and/or physiologically suitable liquid or buffer known in the art. For example, the contrast agent may be contained in water, physiological saline, balanced salt solutions, buffers, aqueous dextrose, glycerol or the like. In certain embodiments, the contrast agent may be administered with agents that may stabilize and/or enhance delivery of the contrast agent to the target site. For example, the contrast agent may be administered with a detergents, wetting agents, emulsifying agents, dispersing agents or preservatives.

In certain embodiments, the contrast agent is administered locally or systemically. Methods of administering include, but are not limited to, intradermal, intramuscular, intraperitoneal, intravenous, subcutaneous, intranasal, rectal, vaginal, and oral routes. The contrast agent may be administered by any convenient route, for example by infusion or bolus injection, by absorption through epithelial or mucocutaneous linings (e.g., oral mucosa, vaginal, rectal and intestinal mucosa, etc.) and may be administered together with other biologically active agents. In certain embodiments, administering the contrast agent includes injecting the contrast agent into a subject at the target site of interest, such as in a body cavity or lumen. In other embodiments, the administering includes providing the contrast agent in an ingestible formulation that a subject may orally ingest to provide the contrast agent at a desired target site, such as a target site in the digestive tract.

In certain embodiments, the method includes obtaining an ultrasound image of a target site in the subject. In some cases, the method includes transmitting an ultrasound signal from an ultrasound transmitter to the target site, and receiving ultrasound data at a receiver. The ultrasound data may be analyzed using a processor, such as a processor configured to analyze the ultrasound data and produce an ultrasound image from the ultrasound data. In certain embodiments, the ultrasound data detected by the receiver includes an ultrasound signal (e.g., an ultrasound signal reflected by the target site of the subject). In certain instances, the method includes applying a set of imaging pulses from an ultrasound transmitter to the target site, and receiving ultrasound signal at a receiver. In certain instances, the ultrasound signal detected by the receiver includes an ultrasound echo signal Additional aspects of ultrasound systems and methods are found, for example, in U.S. Pat. Nos. 6,749,570, 6,705,992, 6,500,118, 6,368,279, and 6,071,240, the disclosures of each of which are incorporated herein by reference.

Methods for performing ultrasound imaging are known in the art and may be employed in methods of the invention. In certain aspects, an ultrasound transducer, which comprises piezoelectric elements, transmits an ultrasound imaging signal (or pulse) in the direction of the target site. Variations in the acoustic impedance (or echogenicity) along the path of the ultrasound imaging signal causes backscatter (or echo) of the imaging signal, which is received by the piezoelectric elements. The received echo signal is digitized into ultrasound data and displayed as an ultrasound image. Conventional ultrasound imaging systems comprise an array of ultrasonic transducer elements that are used to transmit an ultrasound beam, or a composite of ultrasonic imaging signals that form a scan line. The ultrasound beam is focused onto a target site by adjusting the relative phase and amplitudes of the imaging signals. The imaging signals are reflected back from the target site and received at the transducer elements. The voltages produced at the receiving transducer elements are summed so that the net signal is indicative of the ultrasound energy reflected from a single focal point in the subject. An ultrasound image is then composed of multiple image scan lines.

In certain embodiments, the method includes obtaining a first ultrasound data (e.g., echo signal) of the target site, and analyzing the first ultrasound data (e.g., echo signal) to produce an ultrasound image of the target site. The ultrasound data (e.g., echo signal) may be obtained using a standard ultrasound device, or may be obtained using an ultrasound device configured to specifically detect the contrast agent used. Obtaining the ultrasound data (e.g., echo signal) may include detecting the ultrasound signal (e.g., echo signal) with an ultrasound detector.

In certain embodiments, the transducer frequencies used for imaging are 4 MHz or more, 5 MHz or more, 10 MHz or more, or 20 MHz or more, or 30 MHz or more, or 40 MHz or more, or 50 MHz or more. For example, an ultrasound data is obtained by applying to the target site an ultrasound signal at an imaging frequency from 4 to 20 MHz, such as 4.8 MHz to 17 MHz. In certain embodiments, the frequency is selected so as to maximize the contrast generated by the administered gas vesicles.

In certain embodiments, the method includes administering to a subject a contrast enhancing agent to improve the ultrasound image. Ultrasound contrast agents rely on the large acoustic impedance difference between the contrast agent and the surrounding environment. For example, the ultrasound contrast agent may be in an aqueous environment and may comprise a gas. The large difference in acoustic impedance between gas and liquid increases the acoustic backscatter and thus a stronger ultrasound signal is reflected back to the receiver.

In certain embodiments, the method further includes non-linear imaging. By “non-linear”, it is generally meant that the output from a system is not a linear transformation, or function, of the input to the system. In the case of ultrasound imaging, an echo signal that is not a linear function of the imaging signal emitted by the transducer may be said to be non-linear. For example, a method of non-linear ultrasound imaging may include applying a set of ultrasound imaging pulses to the target site and detecting echo signals such that non-linear signals from the gas vesicles are amplified. This may be accomplished by, for example, transmitting at one frequency and receiving at its harmonic or sub-harmonic frequencies. In these instances, non-linear oscillations in the ultrasound contrast agents produce harmonic backscatter of the ultrasound imaging signal. Alternatively, this may be accomplished by pulse inversion sequences. Pulse inversion sequences comprise of successive ultrasonic pulses with opposite signs that are emitted from the ultrasound transducer. Subtracting the successive reflected signals cancels out the linear echo signals and amplifies the non-linear echo signals. Such imaging approaches are described in further detail in, for example, Foster, F. S., Pavlin, C. J., Harasiewicz, K. A., Christopher, D. A. & Turnbull, D. H. Advances in ultrasound biomicroscopy. Ultrasound Med Biol 26, 1-27 (2000), the disclosure of which is incorporated herein by reference.

In certain embodiments, the method further includes disrupting a vesicle wall of the gas vesicles. For example, the method may include applying a pressure to the gas vesicles sufficient to disrupt a vesicle wall of the gas vesicles. In some cases, the pressure may be provided by applying an ultrasonic pulse to the target site sufficient to disrupt a vesicle wall of the gas vesicles. As described above, the ultrasonic pulse may be sufficient to collapse the gas vesicles such that the gas vesicles do not have a substantial contrast-enhancing effect. In these cases, the method may further include obtaining a second ultrasound signal of the target site, and analyzing the first ultrasound signal and the second ultrasound signal (e.g., post-collapse ultrasound signal) to produce an ultrasound image of the target site. For example, the first and second ultrasound signals may be analyzed respectively to produce a first and second ultrasound images, respectively. In some cases, the first and second ultrasound signals may be analyzed to produce a composite image of the first and second ultrasound signals. For instance, the composite image may be a difference image of the first and second ultrasound signals. As described above, the image obtained after the gas vesicles have been collapsed may not have a substantial contrast-enhancing effect, and as such, a difference image may facilitate an increase in the signal to noise ratio in the resulting composite image.

In some embodiments, the method includes the uniplex analysis of a target site of interest in a subject. By “uniplex analysis” is meant that a contrast agent is administered to a target site and the target site is analyzed to detect an ultrasound image of the target site. For example, a single type of contrast agent may be administered to the target site and an ultrasound image of the target site obtained. In some cases, the method includes the uniplex analysis of the target site to determine an ultrasound image of the target site of interest in the subject.

Certain embodiments include the multiplex analysis of two or more contrast agents in a subject. By “multiplex analysis” is meant that the presence two or more distinct contrast agents, in which the two or more contrast agents are different from each other, is determined. For example, contrast agents may be specifically targeted to different target sites in a subject using different specific binding moieties attached to the gas vesicles. In other embodiments, the two or more contrast agents may be different in that they are collapsible at different pressures. For instance, the contrast agents may be derived from different bacteria, and thus may have a different physical structure, and thus be collapsible at different pressures. In these instances, a first and second contrast agent may be administered to a target site in a subject. A first ultrasound signal may be obtained before the application of an external pressure. Then a first ultrasonic pulse may be applied to the target site sufficient to disrupt a vesicle wall of the first gas vesicles (but not sufficient to disrupt a vesicle wall of the second gas vesicles), and a second ultrasound signal may be obtained. A second ultrasonic pulse may be applied to the target site sufficient to disrupt a vesicle wall of the second gas vesicles, and a third ultrasound signal may be obtained. The first, second and third ultrasound signals may be analyzed individually or together to produce individual ultrasound images of the signals or composite images of two or more of the signals.

In other embodiments, the two or more contrast agents may be a genetically engineered variant of a contrast agent. In these embodiments, at least one protein that is a component of, or contributes to the formation of, a contrast agent, such as gas vesicles, may be altered or deleted by genetic engineering such that the genetically engineered protein confers distinct physical properties (e.g., shape and sizes) and thus confers a distinct chemical shift to gas vesicles compared to gas vesicles that do not result from the genetic engineering.

In some instances, the number of contrast agents is greater than 2, such as 3 or more, 4 or more, 5 or more, etc., up to 10 or more, distinct contrast agents. In certain embodiments, the methods include the multiplex analysis of 2 to 10 distinct contrast agents, such as 3 to 10 distinct contrast agents, including 4 to 10 distinct contrast agents.

In certain embodiments, imaging is performed two or more times over a period of time to observe a time-varying event in the subject. For example, after administration of the gas vesicles (e.g., gas vesicles that are functionalized with specific binding groups that facilitate gas vesicle aggregation in the presence of a certain analyte), images may be acquired multiple times to observe one level of signals from gas vesicles in their non-aggregated state, and another level of signals from their aggregated state, the latter being indicative of the presence of the analyte.

### Utility

The subject ultrasound contrast agents and ultrasound imaging methods find use in a variety of different applications where producing an ultrasound image of a target site in a subject is desired. In certain embodiments, the subject ultrasound contrast agents and ultrasound imaging methods find use in uniplex analysis of a target site in a subject. As described above, the subject ultrasound contrast agents and ultrasound imaging methods also find use in the multiplex analysis of a target site in a subject.

Gas vesicle contrast agents thus find use in many molecular imaging applications in cancer, immunology, regenerative medicine and other areas where nanoparticle reporters are desired. Furthermore, the gas vesicles' preferential response at higher frequencies, harmonic signal production and ability to undergo controlled collapse make them useful for high-resolution and contrast-selective nonlinear imaging modes for molecular ultrasonography.

In addition, the ability to image gas vesicles inside cells provides for the above to use gas vesicles as genetically encoded reporters, offering ultrasound an analog of the green fluorescent protein. Gas vesicles are encoded by compact gene clusters (6 kb), two of which may be expressed in Escherichia coli. Bacteria or mammalian cells labeled in this manner provide for non-invasive studies of cellular involvement in processes ranging from infectious disease to organism development. In addition, aggregation-dependent contrast enhancement enables gas vesicles to serve as dynamic molecular sensors for ultrasound, which may be used to sense concentrations and activities of molecules in vivo.

In addition to their use as ultrasound molecular reporters, gas vesicles have a variety of anisotropic shapes, hollow interior, gas permeability, optical scattering, buoyancy, abundance of reactive chemical groups, controlled collapse and possibility of genetic engineering, which facilitates production of gas vesicles with specific properties for the applications described above.

### Kits

Aspects of the present disclosure additionally include kits that include an ultrasound contrast agent. As described above, the ultrasound contrast agent includes a plurality of collapsible gas vesicles. The gas vesicles may include a specific binding moiety attached to a surface of the gas vesicles and configured to specifically bind to a target site in a subject. In certain embodiments, the kit includes a sterile container containing the ultrasound contrast agent.

The kits may further include a buffer. For instance, the kit may include a buffer, such as a sample buffer (e.g., saline, phosphate buffered saline, etc.), and the like. The kits may further include additional components, such as but not limited to, sterile wipes, syringes or other administration devices, and the like.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another means would be a computer readable medium, e.g., portable flash drive, diskette, CD, DVD, Blu-Ray, computer-readable memory, etc., on which the information has been recorded or stored. Yet another means that may be present is a website address which may be used via the Internet to access the information at a removed site. Any convenient means may be present in the kits.

As can be appreciated from the disclosure provided above, embodiments of the present invention have a wide variety of applications. Accordingly, the examples presented herein are offered for illustration purposes and are not intended to be construed as a limitation on the invention in any way. Those of ordinary skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### EXAMPLES

### Example 1

### Methods

### Gas Vesicle Preparation

Anabaena flos-aquae (Ana) was cultured in sterile Gorham's media at room temperature under office fluorescent lighting with an approximately 75% circadian duty cycle. Halobacteria NRC-1 (Halo) was cultured at 37° C. in high-salt medium, under ambient light, according to vendor instructions. GVs were isolated from Ana using hypertonic lysis and centrifugally-assisted flotation. To achieve purity, the harvested GVs were repeatedly resuspended in phosphate-buffered saline (PBS) and re-centrifuged. GVs from Halo were isolated by concentrating the cells through extended floatation and hypotonic lysis, followed by centrifugally assisted floatation as described above. GVs were diluted to experimental concentrations using PBS. To prepare pre-collapsed GVs, GV solutions were loaded into capped plastic syringes and the plunger depressed until the solution became translucent. The concentration of gas vesicles (GVs) was estimated based on pressure-sensitive OD at 500 nm (OD_{500,PS}) due to intact GV light scattering, measured as the difference in optical density between a solution of intact GVs and the same solution of GVs after collapse. The relationship between OD_{500,PS} and protein concentration (in mg/mL) was determined empirically using a BCA protein assay. Literature-based estimates of the molecular weight of the GVs were used to calculate the molar concentration. Values of 564.2±94.2 μM/OD_{500,PS} and 16.1±3.2 pM/OD_{500,PS} were obtained for Ana GVs and Halo GVs, respectively, which were rounded up to 600 and 20 pM/OD_{500,PS}, respectively. Gas volume fractions were estimated using gas volumes of 8.4 μL per mg and 12.3 μL per mg for Ana and Halo GVs, respectively.

### Bio-Functionalization and Aggregation

Ana GVs were biotinylated using EZ-Link Sulfo-NHS-LC-Biotin (Thermo Scientific, Rockford, Ill.) following supplier instructions, with a 10,000-fold molar excess of functionalizing reagent per GV, and purified by floatation. For aggregation experiments, biotinylated GVs were mixed with streptavidin (SA, G-Biosciences, St. Louis, Mo.) at indicated molar ratios and allowed to react for approximately 30 minutes before imaging.

### Ultrasound Imaging

Imaging phantoms were prepared by casting 1% agarose gel (in water) around 96-well PCR reaction tubes, which were removed after solidification. 2× concentrated GV samples were mixed 1:1 with melted 1% agarose, and 100 μL of the mixture was quickly loaded into phantom wells. The same procedure was used to load polystyrene microspheres (0.83% final w/v, 4.78 μm, Spherotech, Lake Forest, Ill.). After all samples were loaded and solidified, an additional 1% agarose was deposited to completely fill the wells and provide a uniform top surface. Imaging was performed using a home-built imaging setup. A 5 MHz, 10 MHz or 20 MHz single element transducer (6.3, 6.3 and 3.2 mm active areas, respectively; 25.4 mm focal distance; Olympus, Waltham, Mass.) was mounted on a computer-controlled 2D translating stage (VelMex, Bloomfield, N.Y.). Transducer output was measured using a hydrophone (HGL-0200, Onda, Sunnyvale, Calif.). Phantoms were placed in a water container such that transducers could be immersed in the water at a distance of approximately 20 mm above the phantom. A programmable pulse generator (AFG3102, Tektronix, Beaverton, Oreg.) and radio frequency amplifier (BT00500-AlphaS-CW, Tomco, Stepney, Australia) were used to drive transducers at specified frequencies with sinusoidal pulse trains of approximately 1 μs. The pre-amplifier function of a pulse-receiver (Panametrics/Olympus 5601A/TT) with high-pass and low-pass filtering at 5 MHz and 75 MHz, connected to an oscilloscope (Infiniivision DSOX2004A, Agilent Technologies, Santa Clara, Calif.; or LeCroy WaveJet 314, Chestnut Ridge, N.Y.) was used to collect ultrasound signals and record them using MATLAB (Mathworks, Natick, Mass.). In situ GV collapse was obtained by repeated pulsing at indicated pressures with a 10 MHz transducer operating at 8 or 8.6 MHz. Specific imaging parameters for each figure are listed in Table 1 below.

### Image Analysis

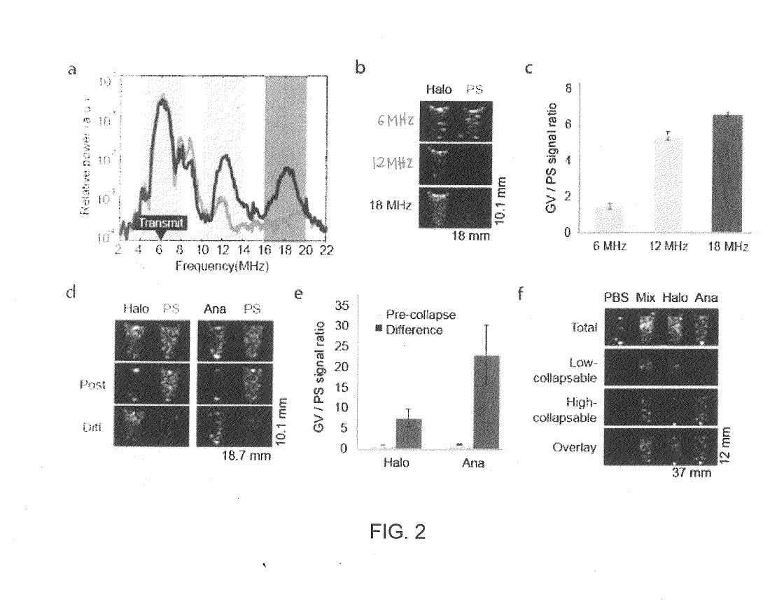

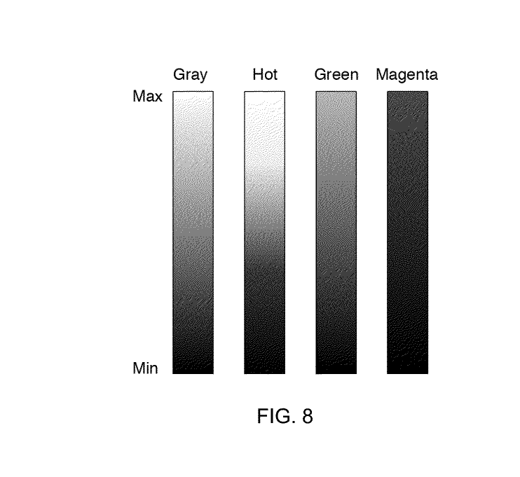

Image analysis was performed in MATLAB. Raw time-domain signals acquired at each scan point were band pass-filtered with symmetric Butterworth filters around the transmit or harmonic frequency, as indicated in Table 1. After filtering, signals were brought back to baseband and time-gain compensation was applied using an empirically determined exponential coefficient. Line intensity profiles were obtained as the absolute value of a five point convolution of the in-phase and quadrature signal components. The resulting line intensity profiles were placed into arrays to generate two-dimensional B-mode images. The time axis was converted to space using the speed of sound. If multiple neighboring lines perpendicular to the B-mode image were acquired, they were added together for averaging. The resulting images were auto-scaled for presentation as indicated in figure legends. Signals from regions of interest in each image were summated and used to generate quantitative plots. Additional processing parameters are listed in Table 1. Color maps used in the images are shown in FIG. 8. Power spectra represent squared absolute values of fast Fourier transforms of raw time-domain signals from regions of interest corresponding to GVs (FIG. 2a. is the average of 48 such spectra).

### Animal Imaging

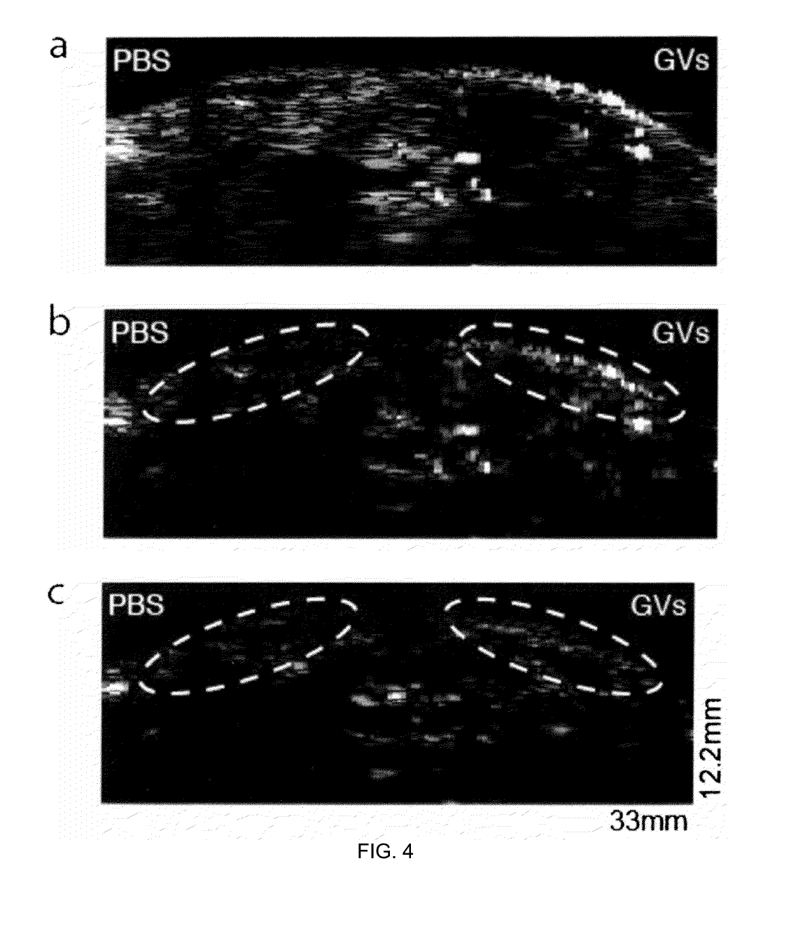

All animal protocols were approved by the Animal Care and Use Committee of the University of California at Berkeley. Female CD-1 mice, maintained under isoflurane anesthesia throughout the experiment, were depilated above the lower abdomen and injected subcutaneously with 150 μL Halo GVs (OD 6) on one side and 150 μL PBS on the other side of the abdomen. Placed on their backs, the mice were fitted with a homemade container allowing a ˜3 cm column of ultrasound gel (Aquasonic, Parker Laboratories, Fairfield, N.J.) to be placed above the injected region. An abdominal constriction was used to reduce breathing-related motion of the lower abdominal region. An ultrasonic transducer was coupled into the gel and scanned or used to apply destructive pulses as described above. To image animal anatomy (e.g., FIG. 4a), the pulse generation function of the pulse-receiver was used in place of the signal generator/amplifier. Additional scan parameters are listed in Table 1.

### Transmission Electron Microscopy (TEM)

TEM images were obtained on a Philips/FEI (Hillsboro, Oreg.) Tecnai 12 microscope operating at 120 kV. 10× diluted GV samples were deposited on a carbon-coated formvar grid and negatively stained with 2% uranyl acetate.

### Statistical Analysis

All error bars and values provided are ±SEM.

TABLE 1
Image Acquisition and Analysis Parameters

| | | | | | Pulse | Aver- | Perpendicular | Analysis | | | | |
|---|---|---|---|---|---|---|---|---|---|---|---|---|
| | Transmit | | Pulse | Peak | Train | ages/ | averages | Center | Analysis | Color | Color map | Color map |
| Figure | Frequency | | Source | Pressure | (us) | line | (distance) | Frequency | Bandwidth | map | max | min |
| 1d - | 17 | Mhz | SG | 175 kPa | 1 μs | 512 | 3 (1 mm) | 17 MHz | 4 MHz | Hot | 40X STD | 0 |
| top | | | | | | | | | | | of PBS well | |
| 1d - | 8.6 | Mhz | SG | 189 kPa | 1 μs | 512 | 3 (1 mm) | 8.6 MHz | 4 MHz | Hot | 10X STD | 0 |
| middle | | | | | | | | | | | of PBS well | |
| 1d - | 4.8 | Mhz | SG | 148 kPa | 1 μs | 512 | 3 (1 mm) | 4.8 MHz | 4 MHz | Hot | 10X STD | 0 |
| bottom | | | | | | | | | | | of PBS well | |
| 1e - | 17 | Mhz | SG | 175 kPa | 1 μs | 512 | 3 (1 mm) | 17 MHz | 4 MHz | Hot | 40X STD | 0 |
| top | | | | | | | | | | | of PBS well | |
| 1e- | 8.6 | Mhz | SG | 189 kPa | 1 μs | 512 | 3 (1 mm) | 8.6 MHz | 4 MHz | Hot | 10X STD | 0 |
| middle | | | | | | | | | | | of PBS well | |
| 1e - | 4.8 | Mhz | SG | 148 kPa | 1 μs | 512 | 3 (1 mm) | 4.8 MHz | 4 MHz | Hot | 10X STD | 0 |
| bottom | | | | | | | | | | | of PBS well | |
| 2b - | 6 | MHz | SG | 98 kPa | 1 μs | 512 | 3 (1 mm) | 6 MHz | 4 MHz | Hot | 0.7X MI | 0 |
| top | | | | | | | | | | | | |
| 2b - | 6 | MHz | SG | 98 kPa | 1 μs | 512 | 3 (1 mm) | 12 MHz | 4 MHz | Hot | MI | 0 |
| middle | | | | | | | | | | | | |
| 2b - | 6 | MHz | SG | 98 kPa | 1 μs | 512 | 3 (1 mm) | 18 MHz | 4 MHz | Hot | MI | 0 |
| bottom | | | | | | | | | | | | |
| 2d - | 8.6 | MHz | SG | 166 kPa | 1 μs | 512 | 3 (1 mm) | 8.6 MHz | 4 MHz | Hot | 0.7X MI | 0 |
| top | | | | | | | | | | | | |
| 2d - | 8.6 | MHz | SG | 166 kPa | 1 μs | 512 | 3 (1 mm) | 8.6 MHz | 4 MHz | Hot | 0.7X MI of | 0 |
| middle | | | | | | | | | | | 2d top | |
| 2d - | Calculated | | | | | | | | | Hot | 0.7X MI of | 0 |
| bottom | | | | | | | | | | | 2d top | |
| 2f - | 8.6 | MHz | SG | 166 kPa | 1 μs | 512 | 3 (1 mm) | 8.6 MHz | 4 MHz | Hot | 0.5 MI | 0 |
| top | | | | | | | | | | | | |
| 2f - | 8.6 | MHz | SG | 166 kPa | 1 μs | 512 | 3 (1 mm) | 8.6 MHz | 4 MHz | Black to | 0.8X MI | 0 |
| magenta | | | | | | | | | | magenta | | |
| 2f - | 8.6 | MHz | SG | 166 kPa | 1 μs | 512 | 3 (1 mm) | 8.6 MHz | 4 MHz | Black to | 0.4X MI | 0 |
| green | | | | | | | | | | green | | |
| 3b | 17 | MHz | SG | 77 kPa | 1 μs | 64 | 1 | 17 MHz | 4 MHz | Hot | MI | 0 |
| 3f | 17 | MHz | SG | 77 kPa | 1 μs | 64 | 1 | 17 MHz | 4 MHz | Hot | MI | 0 |
| 4a - | 10 MHz | | PR | Power | n/a | 512 | 3 (0.1 mm) | 10 MHz | 10 MHz | Gray | 0.2X MI | 0 |
| gray | transducer | | | setting | | | | | | | | |
| | | | | 4 | | | | | | | | |
| 4a - | 6 | MHz | SG | 85 kPa | 1 μs | 512 | 3 (0.1 mm) | 12 MHz | 4 MHz | Black to | 0.35X MI | 0.14X MI |
| green | | | | | | | | | | green | | |
| 4b, c | 6 | MHz | SG | 85 kPa | 1 μs | 512 | 3 (0.1 mm) | 12 MHz | 4 MHz | Hot | 0.7X MI | 0.035X MI |
| Sup. 1 | 17 | Mhz | SG | 175 kPa | 1 μs | 512 | 3 (1 mm) | 17 MHz | 4 MHz | Hot | 100X avg. | 0 |
| | | | | | | | | | | | intensity of | |
| | | | | | | | | | | | PBS | |
| Sup. 2 | 17 | MHz | SG | 175 kPa | 1 μs | 512 | 3 (1 mm) | 17 MHz | 4 MHz | Hot | Scaled to | 0 |
| | | | | | | | | | | | show | |
| | | | | | | | | | | | shadowing | |
| Sup. 3 | 10 MHz | | PR | Power | n/a | 512 | 3 (0.1 mm) | 10 MHz | 10 MHz | Gray | 0.2X MI | 0 |
| a, d, g | transducer | | | setting 4 | | | | | | | | |
| Sup. 3 | 6 | MHz | SG | 85 kPa | 1 μs | 512 | 3 (0.1 mm) | 12 MHz | 4 MHz | Hot | 0.7X MI | 0.035X MI |
| b, c, e, f | | | | | | | | | | | | |
| Sup. 3 | 6 | MHz | SG | 85 kPa | 1 μs | 512 | 3 (0.1 mm) | 12 MHz | 4 MHz | Hot | 0.5X MI | 0.025X MI |
| h, i | | | | | | | | | | | | |

Abbreviations: SG—Signal generator, PR—Pulse receiver, STD—Standard deviation, MI—Maximum intensity

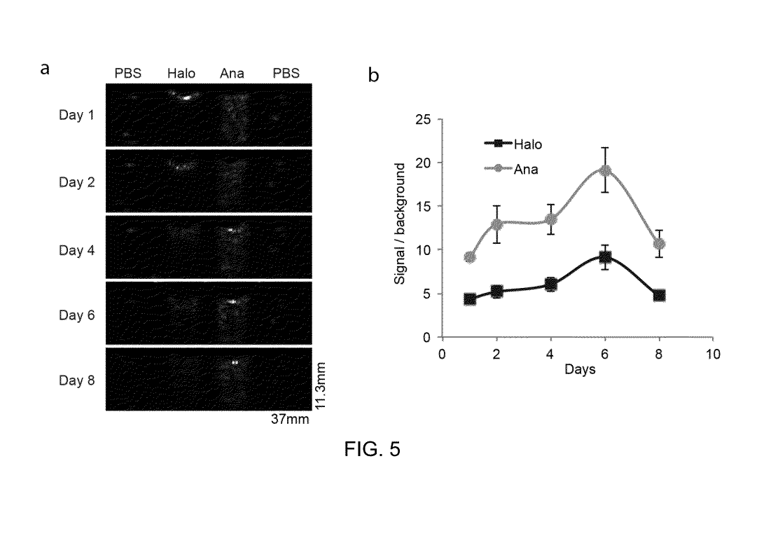

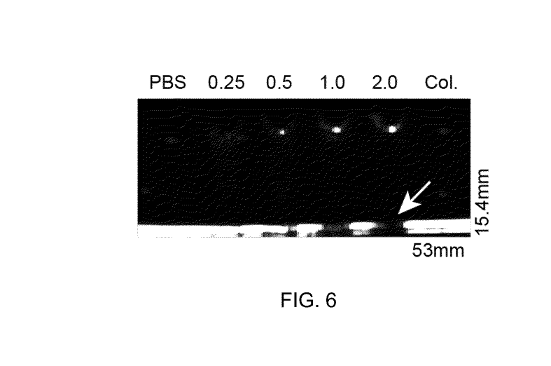

GVs were isolated from Anabaena flos-aquae (Ana) and Halobacterium NRC-1 (Halo) (FIG. 1b-c) and imaged in gel phantoms using a scanning single-element ultrasound imaging system operating at 4.8 MHz, 8.6 MHz and 17 MHz. GVs from both species produced robust contrast relative to buffer controls at optical densities ranging from OD 0.25 to OD 2.0 (FIG. 1d-g), corresponding to nanostructure concentrations of 150 pM to 1.2 nM (Ana) or 5 pM to 40 pM (Halo), and gas volume fractions of approximately 0.01% to 0.1%. GV echogenicity in this configuration was detectable for over one week (FIG. 5). Contrast was strongest at the highest frequency, with OD 2.0 Ana GVs producing 27.0±4.1 greater scattering than buffer controls. The balance of scattering and attenuation differed between Ana and Halo GVs. Whereas Ana GV produced backscatter fairly uniformly along the axial dimension (from top to bottom in FIG. 1d), Halo GVs at higher concentrations produced an attenuation effect extending to the bottom of the phantom (FIG. 1e and FIG. 6).

GVs were collapsed by rapidly increasing the hydrostatic pressure past a species-specific critical point ranging from 40 kPa to over 700 kPa4 (FIG. 1b-c). GVs that were collapsed prior to imaging failed to show ultrasound contrast, confirming the echogenic role of their gas compartments (FIG. 1d-e).

Non-linear imaging modes relying on harmonic signals were used to improve the contrast specificity of contrast-enhanced ultrasound. In particular, harmonic backscatter may arise from non-linear oscillations in gas vesicle radius. Experiments were performed to test whether harmonics could be detected from GVs despite their relative inelasticity. Transmitting at 6 MHz, substantial second and third harmonic signals were observed in Halo GVs at 12 MHz and 18 MHz compared to polystyrene microspheres, a linear scattering material (FIG. 2a). Images formed by processing Halo GV signals through band-pass filters centered at the second and third harmonic frequencies led to 3.7-fold and 4.6-fold GV contrast enhancement relative to the linear reference (FIG. 2b-c). To further increase GV-specific contrast, pressure-induced GV collapse was used. Halo and Ana GVs were imaged before and after a destructive scan at 650 kPa (FIG. 2d). The GV signal was mostly eliminated by collapse, enabling the generation of subtraction images with 10-fold and 22-fold improved Halo and Ana GV contrast, respectively, relative to uncollapsible polystyrene (FIG. 2d-e).

The fact that GVs from different species of bacteria have distinct critical collapse pressures allowed multiplexed ultrasound imaging through serial ultrasonic destruction of two or more types of GVs. To test multiplex imaging, serial destruction imaging of phantoms containing Halo GVs (critical collapse pressure 70−150 kPa), Ana GVs (critical collapse pressure 440−605 kPa) or a combination of both (FIG. 2f) was performed. Halo GV signals were determined by subtracting an image collected after low-pressure (300 kPa) collapse from a pre-collapse baseline. Similarly, Ana GV signals were generated by subtracting an image acquired after high-pressure (650 kPa) collapse from a low-pressure collapsed baseline. An overlay of these serial destruction images demonstrated multiplex imaging (FIG. 2f).

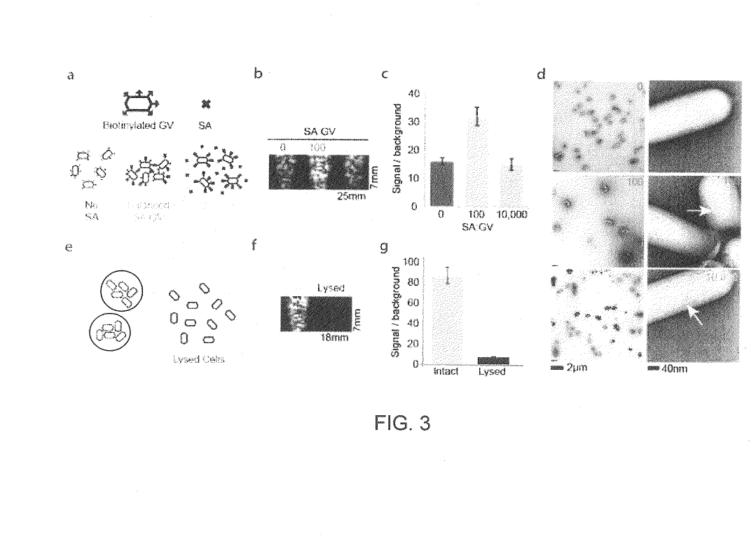

Because GVs were smaller than the wavelengths typically used in ultrasound, assemblies of GV nanoparticles may produce enhanced scattering compared to un-aggregated solutions, enabling GVs to serve as dynamic biomolecular sensors analogous to aggregation-dependent nanoparticle reporters for magnetic resonance imaging (MRI). To test this, the surface of GVs was functionalized with biotin and the GVs were exposed them to various quantities of free streptavidin (SA). In the absence of SA or at SA concentrations sufficient to saturate surface biotins, GVs remained unaggregated; however, at intermediate concentrations, SA mediated GV clustering (FIG. 3a). The formation of aggregates (˜1 μm) in response to SA was confirmed by TEM (FIG. 3d), and at higher magnification it was also possible to discern individual SA molecules on the surface of GVs at the expected relative densities (arrows in FIG. 3d). When nanoparticle assemblies were imaged in ultrasound, the intermediate SA concentration was detected by a two-fold increase in contrast (FIG. 3b-c).

Experiments were performed to test whether GVs expressed inside cells could be used as intracellular reporters or genetic labels. Intact A. flos-aquae was imaged and the resulting contrast was compared to GVs that were released from the same quantity of cells through hypertonic lysis (FIG. 3e-g). The intact cells exhibited a 12-fold stronger signal, indicating that intracellular GVs have the potential to serve as genetically encoded reporters or dynamic indicators of cellular integrity.

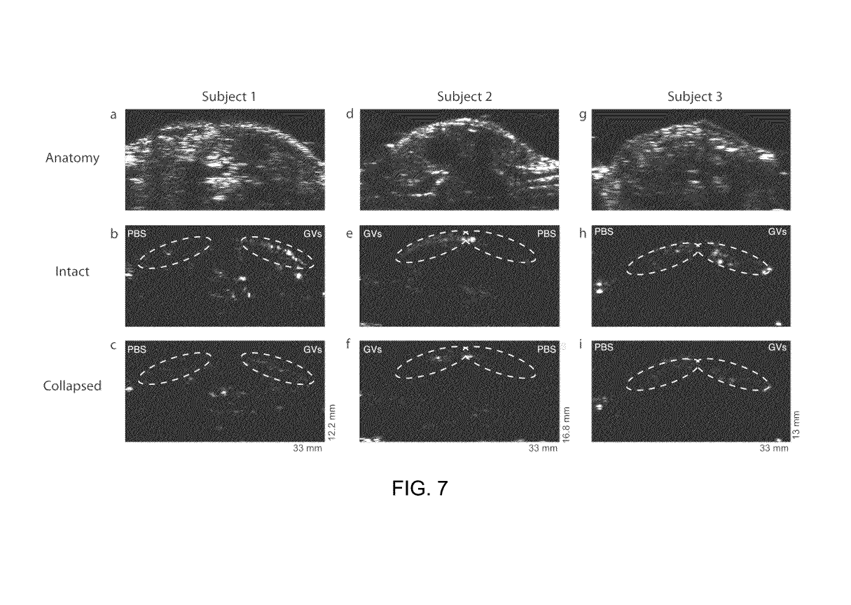

Experiments were performed to determine whether GVs were capable of producing ultrasound contrast in vivo. Halo GVs or a buffer control were injected subcutaneously in the lower abdomen of anesthetized CD-1 mice. To obtain GV-specific images, second-harmonic GV signals were acquired, resulting from transmission at 6 MHz. Significant enhancement on the GV side was observed (FIG. 4a-b). To confirm that GVs were the source of this contrast, destructive pulses were applied, resulting in contrast disappearance (FIG. 4c). These results were consistent across multiple subjects (FIG. 7).

GVs included thin-walled gas nanostructures that produced stable contrast at sub-nanomolar reporter concentrations (gas volume fractions of approximately 0.01% to 0.1%).

Although the foregoing embodiments have been described in some detail by way of illustration and example for purposes of clarity of understanding, it is readily apparent to those of ordinary skill in the art in light of the teachings of the present disclosure that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An ultrasound imaging method comprising:
administering to a subject a contrast agent comprising a plurality of collapsible gas vesicles;
obtaining ultrasound data of a target site; and
analyzing the ultrasound data to produce an ultrasound image of the target site.

2. The method of claim 1, further comprising:
applying an ultrasonic pulse to the target site sufficient to disrupt a vesicle wall of the gas vesicles;
obtaining a second ultrasound data of the target site; and
analyzing the first ultrasound data and the second ultrasound data to produce an ultrasound image of the target site.

3. The method of claim 1, wherein the analyzing comprises producing a first ultrasound image from the first ultrasound data.

4. The method of claim 2, wherein the analyzing comprises producing a second ultrasound image from the second ultrasound data.

5. The method of claim 2, wherein the analyzing comprises producing a composite image of the first and second ultrasound data.

6. The method of claim 1, wherein the obtaining of the first ultrasound data comprises:
applying a first set of imaging pulses; and
detecting a first ultrasound echo signal, such that non-linear signals from the gas vesicles are amplified.

7. The method of claim 1, wherein the obtaining of the first ultrasound data comprises:
applying a first imaging signal at an imaging frequency to the target site; and
detecting a first ultrasound echo signal at one or more of the imaging frequency and a harmonic of the imaging frequency.

8. (canceled)

9. A multiplex ultrasound imaging method comprising:
administering to a subject two or more contrast agents each comprising a plurality of collapsible gas vesicles;
obtaining a first ultrasound data of a target site;
applying a first ultrasonic pulse to the target site sufficient to disrupt a vesicle wall of a plurality of first gas vesicles;
obtaining a second ultrasound data of the target site;
analyzing one or more of the first ultrasound data and the second ultrasound data to produce an ultrasound image of the target site.

10. The method of claim 9, further comprising:
applying a second ultrasonic pulse to the target site sufficient to disrupt a vesicle wall of a plurality of second gas vesicles;
obtaining a third ultrasound data of the target site; and
analyzing one or more of the first ultrasound data, the second ultrasound data and the third ultrasound data to produce an ultrasound image of the target site.

11. The method of claim 9, wherein the analyzing comprises producing a composite image of the first and second ultrasound data.

12. The method of claim 10, wherein the analyzing comprises producing a composite image of the second and third ultrasound data.

13. The method of claim 10, wherein the first ultrasonic pulse is configured to produce a first pressure at the target site, and the second ultrasonic pulse is configured to produce a second pressure at the target site greater than the first pressure.

14. The method of claim 13, wherein the first pressure is sufficient to disrupt the vesicle walls of the first gas vesicles, but is not sufficient to disrupt the vesicle walls of the second gas vesicles.

15. An ultrasound contrast agent comprising:
a plurality of collapsible gas vesicles;
a specific binding moiety attached to a surface of the gas vesicles and configured to specifically bind to a target site in a subject; and
a buffer.

16. The contrast agent of claim 15, wherein the specific binding moiety comprises an antibody configured to specifically bind to the target site in the subject.

17. The contrast agent of claim 15, wherein the gas vesicles have an average cross-sectional diameter of 40 nm to 250 nm.

18. The contrast agent of claim 15, wherein the gas vesicles comprise a gas permeable protein vesicle wall.

19. The contrast agent of claim 15, wherein the gas vesicles are bacterially-derived gas vesicles.

20. The contrast agent of claim 15, wherein the gas vesicles are archaea-derived gas vesicles.

21. The contrast agent of claim 15, wherein the gas vesicles are heterologously expressed in bacterial or mammalian cells.

22-24. (canceled)


SEQUENCE LISTING (1 sequence)

SEQ ID NO: 1 - protein, 71 aa, Anabaena flos-aquae
MAVEKTNSSSSLAEVIDRILDKGIVVDAWVRVSLVGIELLAIEARIVIASVETYLKYAEAVGLTQSAAMPA